(19) United States
(12) Patent Application Publication
(10) Pub. No.: US 2023/0414415 A1
(43) Pub. Date: Dec. 28, 2023

(54) SLEEP MASK WITH BLUE LIGHT BLOCKING LENSES

(71) Applicant: Marian FOX, Monument, CO (US)

(72) Inventor: Marian FOX, Monument, CO (US)

(21) Appl. No.: 17/851,354

(22) Filed: Jun. 28, 2022

(51) Int. Cl. A61F 9/04; B29D 11/00

(52) U.S. Cl. CPC A61F 9/04; B29D 11/00644

(57) ABSTRACT

A sleep mask with blue light blocking lenses includes a soft body member having at least one lens configured to block blue light disposed therein. The mask is configured for comfort and secures over the orbital region of a wearer's face. The mask may include a padding to maintain the at least one lens away from contact with the wearer, even when a wearer lies face down wearing the mask. The at least one lens may be rendered of a flexible, non-rigid, translucent polymer. The wearer may therefore wear the mask while viewing screens in bed without exposure to blue light stimulating wakefulness

### TO ALL WHOM IT MAY CONCERN

Be it known that I, Marian Fox, a citizen of the United States, have invented new and useful improvements in a sleep mask with blue blocking lenses as described in this specification.

### BACKGROUND OF THE INVENTION

Various types of sleep masks are known in the art. All present an opaque surface wearable over the eyes of a wearer to prevent light from reaching the wearer's eyes and assist sleeping in lighted conditions. Sleep masks seen in the present state of the art do not accommodate open eyes, but are devised to be worn with the eyes closed. Such masks typically contact the closed eyelids of the wearer and cover the face above the cheeks and along the forehead of the wearer to present an opaque barrier that prevents passage of light therethrough.

Blue light blocking lenses are also known in the art. However, such lenses are typically mounted in eyewear for use during waking hours, when working and alert. Such lenses are typically mounted in rigid frames for support upon the bridge of the nose and ears in like manner of eyewear seen in the art.

What is needed is a novel combination of such art, wherein pliable blue light blocking lenses are combined into a sleep mask to enable a user to view screens and peripheral devices in bed without stimulating wakefulness due to blue light exposure. The present invention, then, enables a wearer to fall asleep while watching television, say, or to otherwise wear the mask with eyes open in viewing screens on peripheral and electronic devices before bedtime without interfering with the person's sleep cycles. Further, the present invention accommodates comfort when the wearer falls asleep wearing the mask.

### FIELD OF THE INVENTION

The present invention relates to a sleep mask with blue light blocking lenses devised to present a comfortable means of blocking blue light to prevent stimulating wakefulness when viewing screens of peripheral and electronic devices when lying down, or prior to falling asleep.

### SUMMARY OF THE INVENTION

The term “blue light”, as used herein throughout, is taken to mean light of wavelengths in the range of 250 nm to 650 nm and may encompass nonvisible ultraviolet light.

Blue light is known to interfere with human sleep cycles and circadian rhythms. Due to these wavelengths of light scattering during daytime, the human species has evolved to respond to blue light stimulus by wakefulness. However, in the modern era, it has become increasingly common for people to view peripheral devices such as smart phones and tablets in bed prior to falling asleep. Peripheral devices such as these, as well as televisions and other luminous electronic devices presenting images and display, frequently emit light wavelengths across the visible spectrum, including blue light. Such activity, then, may impact a person's ability to fall asleep due to the blue light emitted from such devices. Rather than winding down, then, viewing peripheral devices in bed may excite wakefulness due to blue light stimulation.

The present sleep mask with blue light blocking lenses, therefore, has been devised to enable a comfortable means of sleeping while wearing a mask that is capable of filtering blue light from the eyes. Most blue light blocking eyewear are rigid and non-conformable to the face. Should a person fall asleep wearing such eyewear, they often are awoken by the discomfort caused when the eyewear is pressed against their face. The present invention, then, allows a person to don the sleep mask with blue light blocking lenses, to watch television in bed, for example, or interact with a peripheral device (such as a tablet or smart phone, say) and then fall asleep without the risk of being awoken when sleeping with the mask on. The mask is comfortable and engineered to maintain the lenses removed from contact with the wearer's face. Thus, the mask may be worn with eyes open as well as with eyes closed and a wearer can fall asleep while filtering blue light from reaching the eyes.

The present sleep mask with blue light blocking lenses, therefore, includes a soft body member configured to fit comfortably over the face of a wearer. In an example embodiment contemplated herein, the soft body member is rendered from a soft and conformable fabric that is comfortable to wear and yielding when pressed against the wearer's face so that sleeping facedown with the mask worn is not uncomfortable. Example fabrics from which the body member may be made include silk, cotton, hemp, satin, velvet, and other soft, conformable fabrics that are comfortable to wear. Additional non-fabric materials, such as soft and yielding polymers are also contemplated as within scope of this disclosure.

The present sleep mask with blue light blocking lenses further includes a pair of lenses configured to filter out blue light. The lenses may be pliable, rendered of a transparent polymer that is non-rigid and capable of filtering out certain wavelengths of light, specifically shorter wavelengths of light such as blue light. Such materials include polycarbonate and Trivex® for example, but the lenses contemplated may be more pliable and flexible than polycarbonate or Trivex®.

A soft or pliant strap member is disposed to secure the mask overtop the wearer's face. In some embodiments contemplated herein, the strap may be elasticized and disposed interior to a soft fabric sheath. In other embodiments, the strap may be nonelastic such as a ribbon or other fabric that is attachable together behind the head of the wearer. Further, the mask may include a padding centrally disposed proximal an upper edge of the body member and disposed to contact at least the bridge of the wearer's nose. The padding enables the mask to fit softly and snugly against the wearer's face, even when pressured against the wearer such as when the wearer is lying face down while wearing the mask. The padding prevents direct contact of the lenses with the wearer. Thus, the sleep mask conforms to the wearer's face as a soft surface against which the wearer is not discomforted when placing pressure against the mask when worn.

Thus has been broadly outlined the more important features of the present sleep mask with blue light blocking lenses so that the detailed description thereof that follows may be better understood and in order that the present contribution to the art may be better appreciated.

Objects of the present sleep mask with blue light blocking lenses, along with various novel features that characterize the invention are particularly pointed out in the claims forming a part of this disclosure. For better understanding of the sleep mask with blue light blocking lenses, its operating advantages and specific objects attained by its uses, refer to the accompanying drawings and description.

### BRIEF DESCRIPTION OF THE DRAWINGS

FIG. 1 is a rear diagonal elevation view of an example embodiment.

FIG. 2 is a front diagonal elevation view of an example embodiment.

FIG. 3 is a front elevation view of an example embodiment.

FIG. 4 is a rear elevation view of an example embodiment.

FIG. 5 is a ride side elevation view of an example embodiment.

FIG. 6 is a left side elevation view of an example embodiment.

FIG. 7 is a top elevation view of an example embodiment.

FIG. 8 is a bottom elevation view of an example embodiment.

### DETAILED DESCRIPTION OF THE DRAWINGS

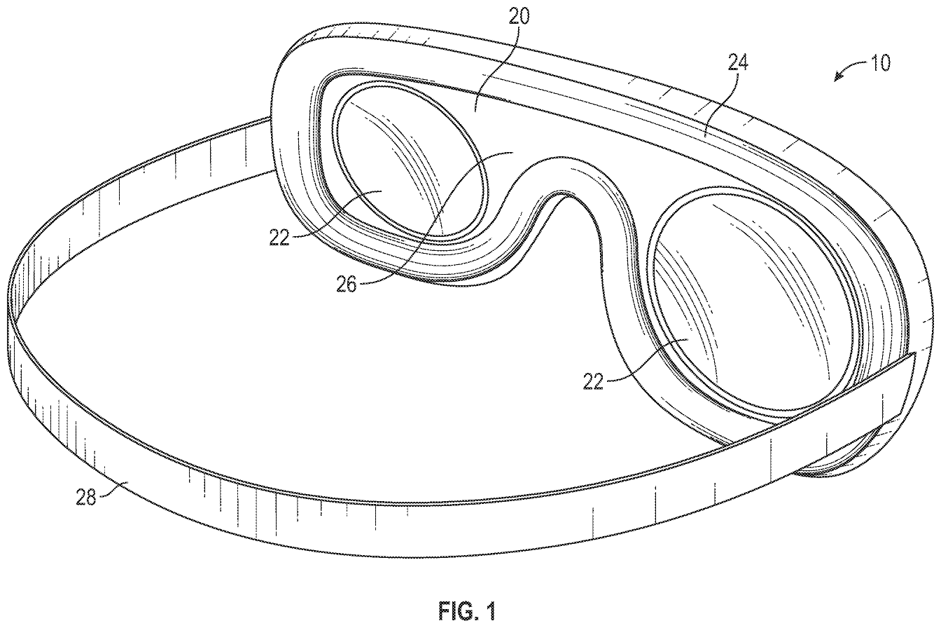

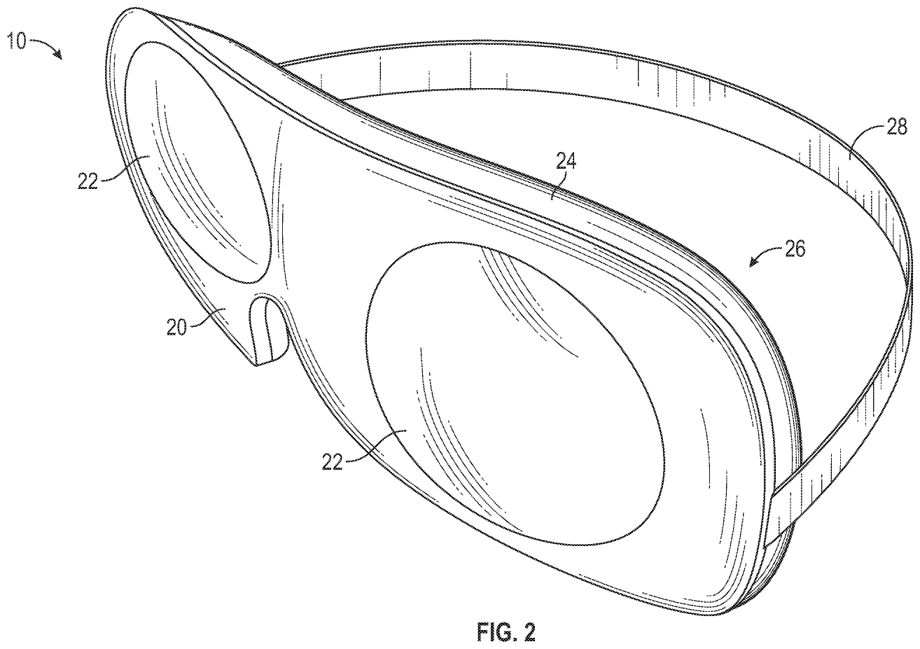

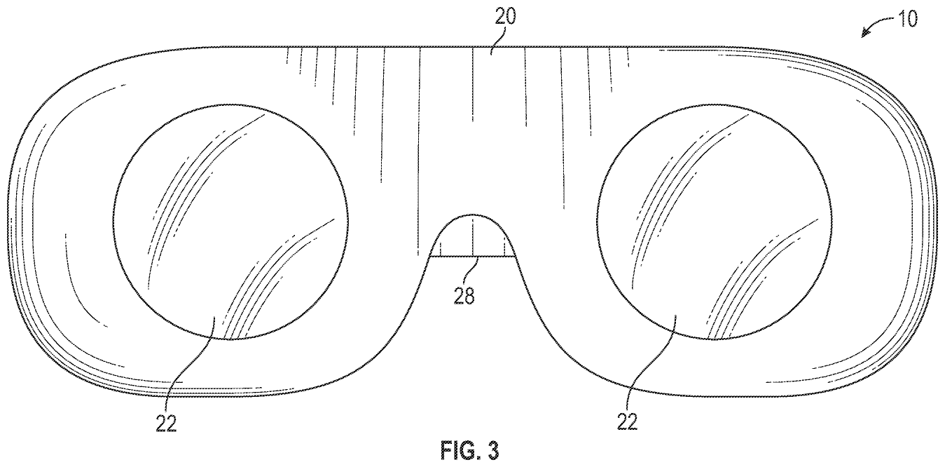

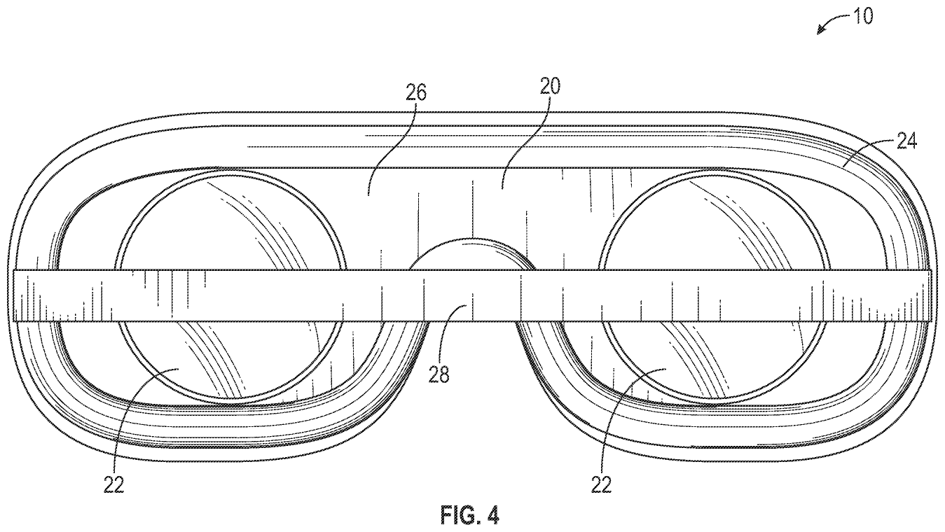

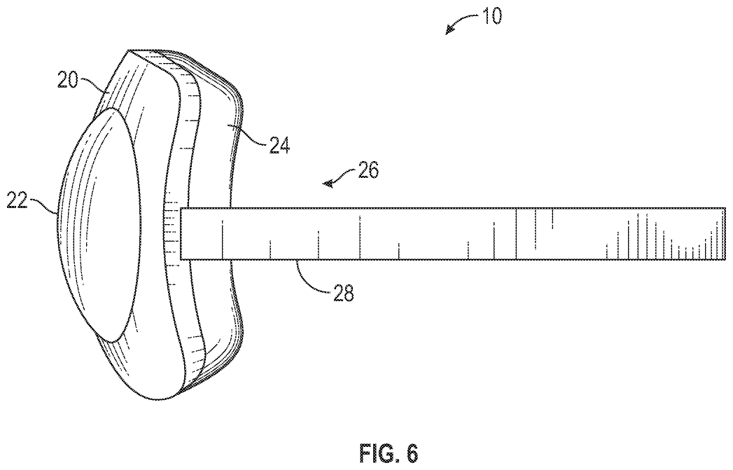

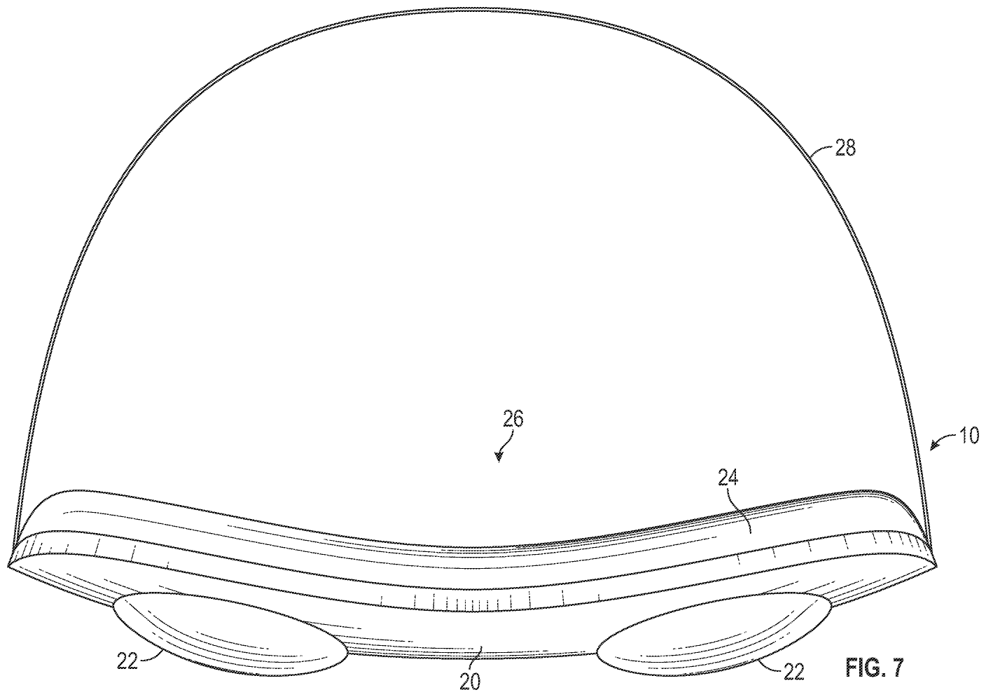

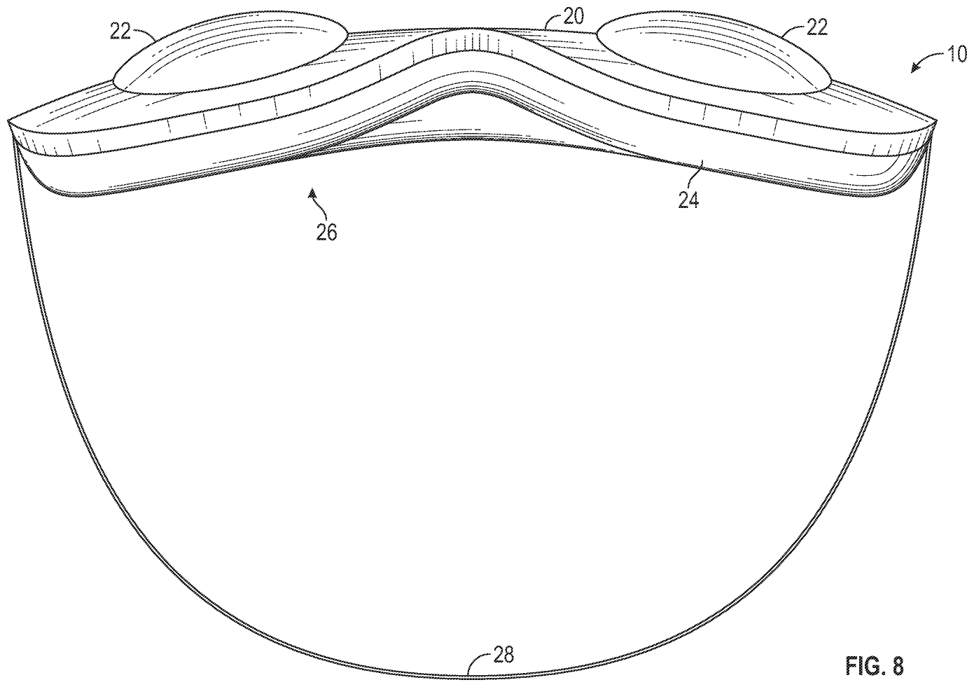

An example embodiment of the instant sleep mask with blue blocking lenses 10 is shown in FIGS. 1 through 8. Soft body member 20 is configured to comfortably fit upon the face of a wearer, over the orbital region around the wearer's eyes and the glabella and bridge of the nose. Soft body member 20 may be rendered of any soft or pliant or conformable material, including fabrics and polymers and other materials. Soft body member 20 includes pair of lenses 22. Pair of lenses 22 is recessed into body member 20 to prevent contact with a wearer's eyelashes when the sleep mask 10 is worn. Pair of lenses 22 is configured to filter out blue light and may comprise a flexible, translucent polymer, capable of bending or flexing at least to some degree to prevent rigidity of the mask 10 when worn. Alternatively, pair of lenses 22 may be glass or other translucent material or polymer.

Soft body member 20 includes padding 24 disposed, in the example embodiment illustrated, perimetrically around interior surface of mask 10. Padding 24 may serve to maintain the mask 10 comfortably in position in contact with the wearer's forehead, orbital region around the eyes, the temples, and/or cheeks. Padding 24 ensures pair of lenses 22 is maintained spaced apart from contact with the wearer a sufficient distance to enable the wearer to open and close his or her eyes without contacting pair of lenses 22 or other parts of soft body member 20. Padding 24 may be compressible to ensure comfort when the wearer falls asleep with the mask on while maintaining sufficient capacity to keep the pair of lenses 22 from coming into contact with the wearer. Alternative or other configurations of padding 24 are contemplated as within the instant application's disclosure, including padding 24 that does not surround the periphery of soft body is member 20, but padding 24 incorporated at select positions upon interior surface 26 to maintain contact with a wearer while keeping pair of lenses 22 suitably removed from contact with the wearer. Such partial padding 24 may include padding disposed to contact the wearer at the glabella or interciliary space above the bridge of the nose, for example, or padding to contact key and select positions around the orbital region of the wearer.

Mask 10 is secured to the wearer by soft or pliable strap member 28. Strap member 28 may be fastenable or elasticized or otherwise securable about the head of a wearer. Strap member 28 may be disposed interior to an external fabric sheath. Strap member 28 is soft and pliable to ensure comfort when worn and to remain comfortable when a wearer sleeps wearing the mask 10. Strap member 28 may be manufactured of elastic or inelastic material, and may include fastenable elements, or be disposed interior to a soft and comfortable sheath. Strap member 28 may be rendered of fabric, polymer, or other soft and conformable material.

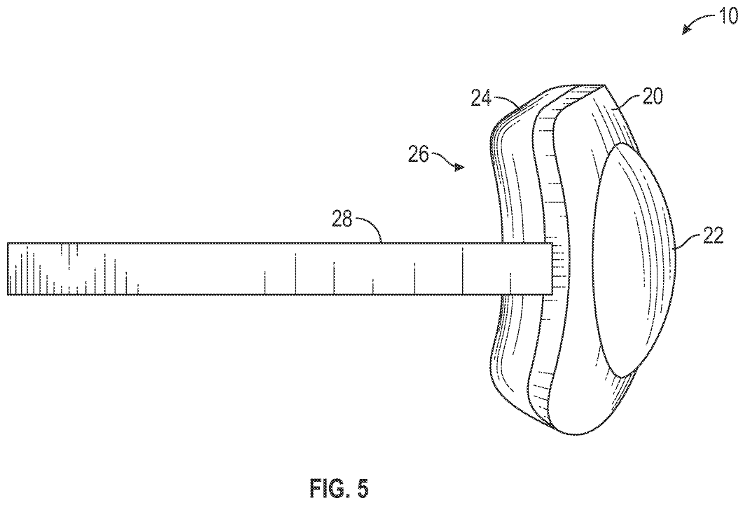

As is shown particularly in FIGS. 5 through 8, pair of lenses 22 may be shaped or mounted convexly in soft body member 20 to present a concavity on interior surface of body member 20 to thereby further prevent contact with the orbital region of the wearer.

What is claimed is:

1. A sleep mask with blue light blocking lenses comprising:
a soft body member configured to fit comfortably over the orbital region of the face of a wearer;
a pair of lenses configured to filter out blue light disposed in the soft body member; and
a soft or pliant strap member disposed to secure the mask upon the wearer's face;
wherein the sleep mask conforms to the wearer's face as a soft surface against which the wearer is not discomforted when placing pressure against the mask when worn.

2. The sleep mask with blue light blocking lenses of claim 1 wherein each of the pair of lenses is pliable and non-rigid.

3. The sleep mask with blue light blocking lenses of claim 2 wherein the strap member comprises two, non-elastic members which are fastenable together.

4. The sleep mask with blue light locking lenses of claim 2 wherein the mask further comprises a padding disposed to contact at least the interciliary space of the wearer whereby the mask softly fits covering the wearer's orbital region even when pressured against the wearer's face such as when the wearer is lying facedown while wearing the mask.

5. The sleep mask with blue light blocking lenses of claim 2 wherein the strap member is elastic.

6. The sleep mask with blue light blocking lenses of claim 5 wherein the strap member is disposed interior to a fabric sheath.

7. The sleep mask with blue light blocking lenses of claim 6 wherein the mask further comprises a padding disposed to contact at least the interciliary space of the wearer whereby the mask softly fits against the wearer's face even when pressured against the wearer's face such as when the wearer is lying facedown while wearing the mask.

8. The sleep mask with blue light blocking lenses of claim 6 wherein the mask further comprises a padding disposed to at least selectively contact the wearer around the orbital region of the wearer's face.
